(19) United States
(12) Patent Application Publication
(10) Pub. No.: US 2024/0197463 A1
(43) Pub. Date: Jun. 20, 2024

(54) ORTHOPAEDIC SURGICAL SYSTEM AND METHOD FOR INSTALLING CEMENT RESTRICTOR COMPONENTS IN A DIRECT ANTERIOR APPROACH HIP REPLACEMENT SURGICAL PROCEDURE

(71) Applicant: DePuy Ireland Unlimited Company, Ringaskiddy (IE)

(72) Inventors: James A. Anderson, York (GB); J. Bohannon Mason, Charlotte, NC (US); Neil D. Watkins, Lymm (GB)

(21) Appl. No.: 18/082,768

(22) Filed: Dec. 16, 2022

(51) Int. Cl. A61F 2/02; A61F 2/32

(52) U.S. Cl. CPC A61F 2/02; A61F 2/32

(57) ABSTRACT

An orthopaedic surgical system for use in a direct anterior approach orthopaedic surgical hip replacement procedure on a patient's femur includes a proximal guide instrument for guiding a flexible insertion instrument to install a cement restrictor component. A method of installing a cement restrictor component during performance of a direct anterior approach orthopaedic surgical hip replacement procedure is also disclosed.

### TECHNICAL FIELD

The present disclosure relates generally to orthopaedic instruments, and particularly to orthopaedic instruments for use in hip replacement surgery.

### BACKGROUND

Joint arthroplasty is a well-known surgical procedure by which a diseased and/or damaged natural joint is replaced by a prosthetic joint. The prosthetic joint may include a prosthesis that is implanted into one or more of the patient's bones. Many hip prostheses include a femoral prosthesis that is implanted into a patient's femur. A femoral prosthesis typically includes an elongated stem component that is cemented in the medullary canal of the patient's femur and a spherically-shaped head component that bears against the patient's acetabulum or a prosthetic replacement acetabular cup.

Some hip replacement procedures are performed using a direct anterior approach. When using the direct anterior approach, the surgeon does not have line-of-sight access to the intramedullary canal of the patient's femur. This limited access and visibility makes it difficult for the surgeon to install certain components, such as cement restrictor components, using instruments designed for other approaches (e.g., a posterior approach).

### SUMMARY

According to one aspect, a method of installing a cement restrictor component during performance of a direct anterior approach orthopaedic surgical hip replacement procedure on a patient's femur includes sliding an elongated shaft of an insertion instrument through an elongated bore of a proximal guide instrument. The insertion instrument has a handle on its proximal end. The proximal guide instrument has an elongated post through which the bore extends and a neck extending medially from the elongated post. The cement restrictor component is secured onto a distal end of the insertion instrument so as to slidably capture the proximal guide instrument on the elongated shaft between the handle of the insertion instrument and the cement restrictor component. Thereafter, the cement restrictor component is advanced through a surgically-prepared proximal end of the patient's femur and into an intramedullary canal of the patient's femur. The proximal guide instrument is then slid along the shaft of the insertion instrument such that the post and the neck of the proximal guide instrument are frictionally secured in the surgically-prepared proximal end of the patient's femur. Thereafter, the shaft of the insertion instrument is slid relative to the secured proximal guide instrument such that the cement restrictor component is advanced distally into the intramedullary canal of the patient's femur to a desired depth.

The method may further include unsecuring the cement restrictor component from the distal end of the insertion instrument once the cement restrictor component has been advanced to the desired depth, and sliding the shaft of the insertion instrument relative to the secured proximal guide instrument such that the distal end of the insertion instrument is advanced proximally out of the intramedullary canal of the patient's femur.

In an embodiment, the cement restrictor component is threaded onto a threaded end of the shaft of the insertion instrument.

In an illustrative embodiment, a superior end of the post of the proximal guide instrument defines a planar surface, and the shaft of the insertion instrument has a depth stop secured thereto at a location between the handle of the insertion instrument and the distal end of the insertion instrument. A distal surface of the depth stop defines a planar surface. The shaft of the insertion instrument is slid relative to the secured proximal guide instrument until the planar surface of the depth stop engages the planar surface of the proximal guide instrument.

In one example, the cement restrictor component is embodied as a cement restrictor trial component that is advanced distally into the intramedullary canal of the patient's femur to the desired depth.

In another example, the cement restrictor component is embodied as a cement restrictor implant component that is advanced distally into the intramedullary canal of the patient's femur to the desired depth.

In an illustrative embodiment, the shaft of the insertion instrument is bowed during advancement of the cement restrictor component.

In another aspect, a method of installing a cement restrictor implant component during performance of a direct anterior approach orthopaedic surgical hip replacement procedure on a patient's femur includes sliding an elongated shaft of an insertion instrument through an elongated bore of a proximal guide instrument. The insertion instrument has a handle on its proximal end. The proximal guide instrument has an elongated post through which the bore extends and a neck extending medially from the elongated post. The cement restrictor implant component is secured onto a distal end of the insertion instrument so as to slidably capture the proximal guide instrument on the elongated shaft between the handle of the insertion instrument and the cement restrictor implant component. Thereafter, the cement restrictor implant component is advanced through a surgically-prepared proximal end of the patient's femur and into an intramedullary canal of the patient's femur. The proximal guide instrument is then slid along the shaft of the insertion instrument such that the post and the neck of the proximal guide instrument are frictionally secured in the surgically-prepared proximal end of the patient's femur. Thereafter, the shaft of the insertion instrument is slid relative to the secured proximal guide instrument such that the cement restrictor implant component is advanced distally into the intramedullary canal of the patient's femur to a desired depth. The cement restrictor implant component is unsecured from the distal end of the insertion instrument once the cement restrictor implant component has been advanced to the desired depth so as to implant the cement restrictor implant component at the desired depth.

The shaft of the insertion instrument may be slid relative to the secured proximal guide instrument such that the distal end of the insertion instrument is advanced proximally out of the intramedullary canal of the patient's femur.

The cement restrictor implant component may be threaded onto a threaded end of the shaft of the insertion instrument.

In an illustrative embodiment, a superior end of the post of the proximal guide instrument defines a planar surface, and the shaft of the insertion instrument has a depth stop secured thereto at a location between the handle of the insertion instrument and the distal end of the insertion instrument. A distal surface of the depth stop defines a planar surface. The shaft of the insertion instrument is slid relative to the secured proximal guide instrument until the planar surface of the depth stop engages the planar surface of the proximal guide instrument.

In an illustrative embodiment, the shaft of the insertion instrument is bowed during advancement of the cement restrictor implant component.

According to another aspect, an orthopaedic surgical system for use in a direct anterior approach orthopaedic surgical hip replacement procedure on a patient's femur includes a proximal guide instrument having an elongated post with a bore formed therein. The bore extends from a superior end of the elongated post to an inferior end of the elongated post and has a common diameter throughout its entire length. The proximal guide instrument also includes a neck extending medially from the elongated post. The orthopaedic surgical system also includes an insertion instrument having a handle at its proximal end and a threaded distal end. The insertion instrument also includes a flexible elongated shaft extending distally away from the handle to the threaded distal end. The flexible elongated shaft has a diameter that is sized to be slidingly received in the bore of the proximal guide instrument. The orthopaedic surgical system also includes a cement restrictor component configured to be threaded onto the threaded distal end of the insertion instrument.

In one embodiment, the flexible elongated shaft is constructed of a superelastic metal.

In a specific illustrative embodiment, the flexible elongated shaft is constructed of a nickel titanium alloy.

The cement restrictor component may be embodied as a cement restrictor trial component or a cement restrictor implant component.

In an embodiment, the superior end of the post of the proximal guide instrument defines a planar surface, with the flexible elongated shaft of the insertion instrument having a depth stop secured thereto at a location between the handle of the insertion instrument and the threaded distal end of the insertion instrument. A distal-most surface of the depth stop defines a planar annular surface having a diameter that is larger than the common diameter of the bore of the proximal guide instrument.

### BRIEF DESCRIPTION OF THE DRAWINGS

The detailed description particularly refers to the following figures, in which:

FIG. 1 is an elevation view of an orthopaedic surgical system for use in a direct anterior approach orthopaedic surgical hip replacement procedure on a patient's femur;

FIG. 2 is a perspective view of the proximal guide instrument of the orthopaedic surgical system of FIG. 1;

FIG. 3 is a cross-sectional view of the proximal guide instrument of FIG. 2, taken along the line 3-3 of FIG. 2, as viewed in the direction of the arrows;

FIG. 4 is an elevation view of the orthopaedic surgical system of FIG. 1 showing the cement restrictor trial component secured to the insertion tool such that the proximal guide instrument is slidably captured on the flexible elongated shaft of the insertion tool;

FIG. 5 is a cross-sectional view of a patient's femur during a direct anterior approach hip replacement surgical procedure, showing a surgeon introducing a cement restrictor trial component into the intramedullary canal of a patient's femur, note the proximal guide instrument is shown secured to the bone tissue of the proximal end of the patient's femur;

FIG. 6 is a view similar to FIG. 5, but showing the cement restrictor trial component being distally advanced into the intramedullary canal of the patient's femur by use of the insertion instrument under guidance of the proximal guide instrument; and

FIG. 7 is a view similar to FIG. 6, but showing the cement restrictor trial component having reached a desired depth within the intramedullary canal of the patient's femur.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout the specification in reference to the orthopaedic implants or prostheses and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

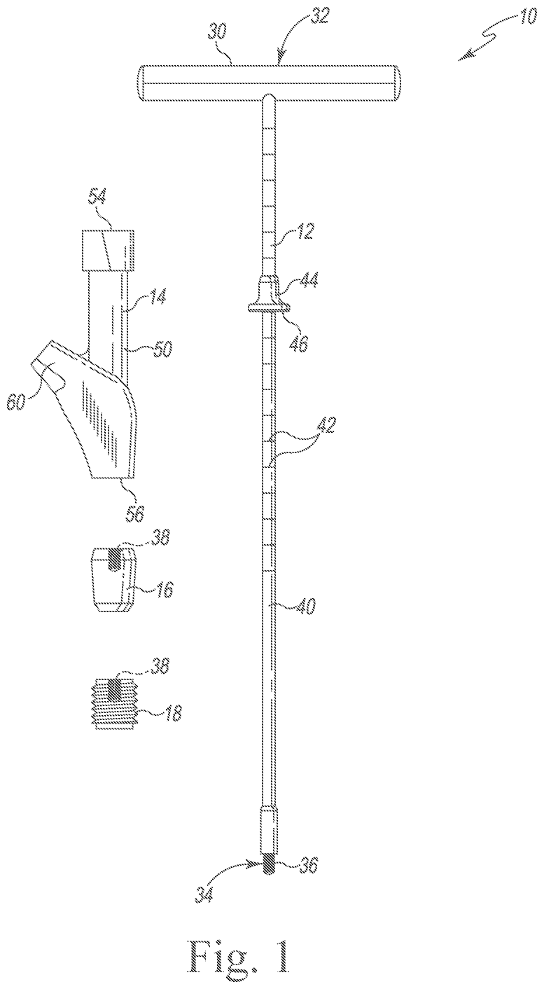

Referring to FIG. 1, an orthopaedic surgical system 10 is illustratively embodied to include an insertion instrument 12, a proximal guide instrument 14, and a pair of cement restrictor components—specifically, a cement restrictor trial component 16 and a cement restrictor implant component 18. As will be discussed below in more detail, the orthopaedic surgical system 10 is used to install the cement restrictor components 16, 18 into an intramedullary canal 24 of a patient's femur 26 during a direct anterior approach surgical hip replacement procedure.

As can be seen in FIG. 1, the insertion instrument 12 is generally T-shaped and has a handle 30 at its proximal end 32. The opposite distal end of the insertion instrument 12 defines a threaded tip 34 with number of male threads 36 formed therein. The cement restrictor components 16, 18 have threaded bores 38 formed therein that are sized to threadingly receive the male threads 36 of the threaded distal tip 34 of the insertion instrument 12 to selectively secure the cement restrictor components 16, 18 to the instrument 12.

A flexible elongated shaft 40 extends distally away from the center of the handle 30 towards the threaded distal tip 34 of the insertion instrument 12. The longitudinal axis of the flexible elongated shaft 40 is orthogonal to the longitudinal axis of the handle 30, thus providing the instrument's generally T-shaped configuration. The handle 30, the flexible elongated shaft 40, and the threaded distal tip 34 may be integrally formed with one another, or may be provided as separate components welded or otherwise secured to one another.

To facilitate a direct anterior approach hip arthroplasty, the flexible elongated shaft 40 is constructed of a highly flexible metallic material. In an embodiment, the flexible elongated shaft 40 is constructed of a metal material that exhibits superelasticity properties. Superelasticity is the ability of a metal to undergo large deformations and immediately return to its undeformed shape upon removal of the external load. One superelastic metal that may be used in the construction of the flexible elongated shaft 40 is a nickel titanium alloy (referred to commonly as “Nitonol”). Such a highly deformable metal allows the insertion tool 10 to facilitate the installation of the cement restrictor components 16, 18 “around the corner” of the proximal end 28 of the patient's femur 26 despite not having line-of-sight access to the intramedullary canal 24 during a direct anterior approach hip arthroplasty.

As can be seen in FIG. 1, the flexible elongated shaft 40 has a number of depth marks 42 formed thereon at a locations above the threaded distal tip 34. Each of the depth marks 42 corresponds to a depth in which the cement restrictor components 16, 18 have been advanced into the intramedullary canal 24 of the patient's femur 26 during installation thereof. The flexible elongated shaft 40 of the insertion instrument 12 also has a depth stop 44 secured thereto at a location between handle 30 and the threaded distal tip 34. As will be described in more detail below, the depth stop 44 engages a superior surface of the proximal guide instrument 14 when the cement restrictor components 16, 18 have been advanced into the intramedullary canal 24 of the patient's femur 26 to a desired depth during installation thereof. The distal-most surface 46 of the depth stop 44 defines a planar annular surface having a diameter that is larger than the bore formed in the proximal guide instrument 14 thereby causing the depth stop 44 to engage the superior surface of the proximal guide instrument 14 (as opposed to entering the bore) and thus prevent further advancement of the flexible elongated shaft 40 (and the cement restrictor component 16, 18 secured thereto).

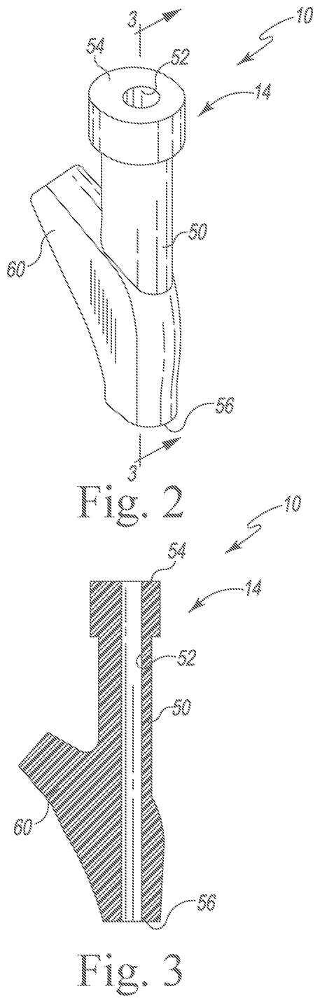

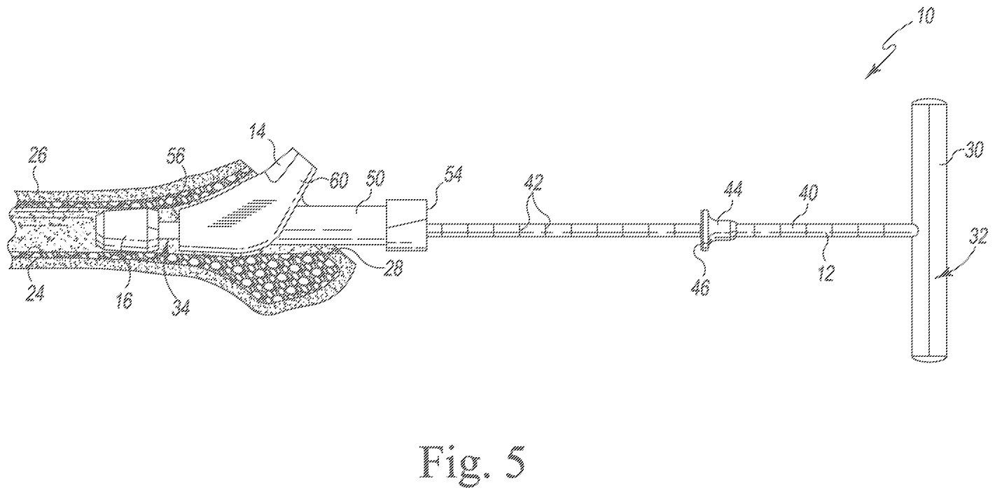

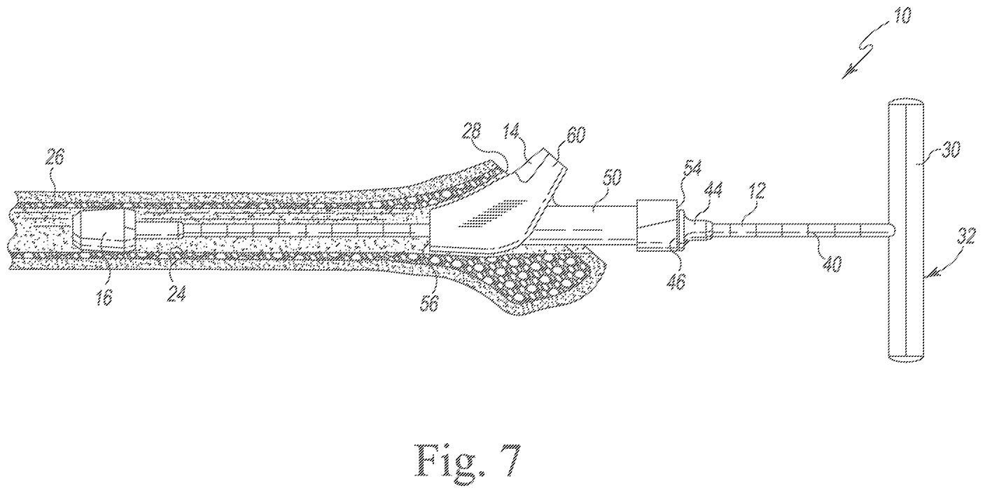

As shown in FIGS. 2 and 3, the proximal guide instrument 14 includes an elongated post 50 having a bore 52 formed therein. As can be seen in FIG. 3, the bore 52 extends from a superior end 54 of the elongated post 50 to an inferior end 56 of the elongated post 50. The bore 52 has a common diameter throughout its entire length. In other words, the diameter of the bore 52 is the same throughout its entire length from its end that opens into the superior end 54 of the elongated post 50 to its end which opens into the inferior end 56 of the post 50. As can be seen in FIGS. 5-7, the diameter of the bore 52 is large enough to allow passage therethrough of the distal tip 34 and the flexible elongated shaft 40, but not the depth stop 44. In particular, the planar annular surface defined by the distal-most surface 46 of the depth stop 44 has a diameter that is larger than the diameter of the bore 52 thereby preventing the depth stop 44 from entering the bore 52 and thus causing the depth stop 44 to instead engage the superior end 54 of the proximal guide instrument 14 so as to prevent further advancement of the flexible elongated shaft 40 (and the cement restrictor component 16, 18 secured thereto).

The proximal guide instrument 14 also includes a curved neck 60 extending medially from the elongated post 50. In such a way, the proximal guide instrument 14 takes on the shape and size of a proximal body component of a femoral hip prosthesis (along with a correspondingly shaped proximal body trial component). The proximal guide instrument 14 may be provided in a range of sizes to correspond with different sizes of femoral implants to be implanted in the patient's femur 26. Across such a range of sizes, the length of the portion of the post 50 that extends above the neck 60 may be varied to adjust the depth in which the cement restrictor components 16, 18 are installed in the intramedullary canal 24 of the patient's femur. In particular, as described above, the superior end 54 of the elongated post 50 defines a planar stop surface that is engaged by the distal-most surface 46 of the depth stop 44 during advancement of the flexible elongated shaft 40 (and thus the cement restrictor component 16, 18 secured thereto) to prevent further movement of the shaft 40. As can be seen in FIGS. 5-7 (and will be discussed in more detail below), the neck 60 of the proximal guide instrument 14 is secured in place within the proximal end 28 of the patient's femur 26 prior to installation of the cement restrictor components 16, 18. By varying the length of the portion of the post 50 that extends above the neck 60, the position of the planar stop surface defined by the superior end 54 of the elongated post 50 is likewise varied relative to the intramedullary canal 24 of the patient's femur 24. As such, the position in which the depth stop 44 of the flexible elongated shaft 40 engages the proximal guide instrument 14 may likewise be varied thus allowing the depth at which the cement restrictor components 16, 18 are installed in the intramedullary canal 24 of the patient's femur 24 to be varied. For instance, a proximal guide instrument 14 with a relatively long post 50 will be engaged by the depth stop 44 earlier than a proximal guide instrument 14 with a relatively short post 50 thereby causing the cement restrictor component 16, 18 to be installed at a shallower depth in the intramedullary canal 24 relative to use of the proximal guide instrument 14 with a relatively short post 50. As such, if a surgeon selects a proximal guide instrument 14 with a short post 50, the cement restrictor component 16, 18 will be installed at a deeper depth in the intramedullary canal 24 than if the surgeon had selected a proximal guide instrument 14 with a long post 50 (and vice versa).

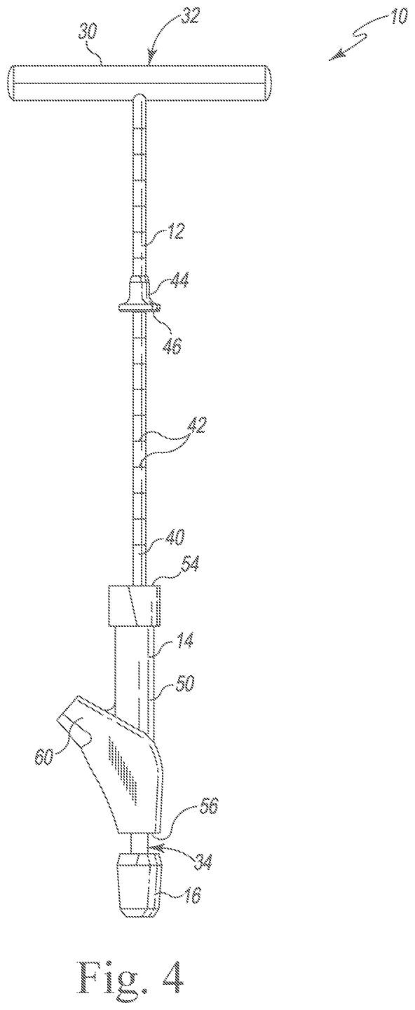

In use, the orthopaedic surgical system 10 may be used by a surgeon to install the cement restrictor components 16, 18 in the intramedullary canal 24 of a patient's femur 26 during a direct anterior approach hip replacement surgical procedure. Typically, the surgeon assembles and installs a trial construct of the femoral prosthesis and trials the fit before implanting the final prosthesis. As such, as shown in FIG. 4, the surgeon initially prepares for installation of the cement restrictor trial component 16. To do so, the surgeon first selects a proximal guide instrument 14 having the desired size from the various available sizes. As noted above, the size of the proximal guide instrument 14 may be selected based on the installation depth desired for the cement restrictor trial component 16. For instance, as described above, if a surgeon selects a proximal guide instrument 14 with a short post 50, the cement restrictor trial component 16 will be installed at a deeper depth in the intramedullary canal 24 than if the surgeon selects a proximal guide instrument 14 with a long post 50 (and vice versa). Hence, the surgeon selects a size of the proximal guide instrument 14 that fits the needs of the specific patient and surgical situation. The surgeon then installs the selected proximal guide instrument 14 on the insertion instrument 12 by sliding the distal tip 34 and the flexible elongated shaft 40 of the insertion instrument 12 through the superior end 54 of the elongated post 50 and into the elongated bore 52 of the proximal guide instrument 14. Once the threaded distal tip 34 exits the bore 52 through the inferior end 56 of the elongated post 50, the cement restrictor trial component 16 is threaded onto the threaded distal tip 34. Doing so captures the proximal guide instrument 14 on the flexible elongated shaft 40 such that it may slide freely along the shaft 40, but is retained on the shaft 40 by the depth stop 44 and the installed cement restrictor trial component 16.

Once the proximal guide instrument 14 and the cement restrictor trial component 16 have been assembled to the insertion instrument 12, as shown in FIG. 5, the surgeon advances the cement restrictor trial component 16 through the surgically-prepared proximal end 28 of the patient's femur 26 and into the intramedullary canal 24. Thereafter, the surgeon slides the proximal guide instrument 14 distally along the flexible elongated shaft 40 and into the surgically-prepared proximal end 28 of the patient's femur 26. The surgeon continues to advance the proximal guide instrument 14 along the shaft 40 until the post 50 and the neck 60 of the instrument 14 are lightly driven into the bone tissue of the surgically-prepared proximal end 28 of the patient's femur 26, as shown in FIG. 5, so as to frictionally secure the proximal guide instrument 14 therein.

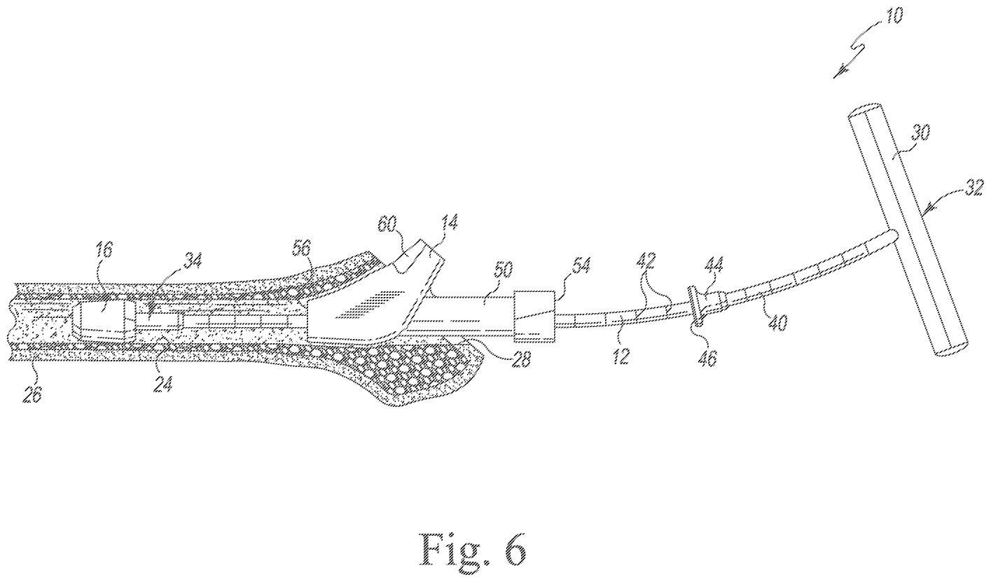

With the proximal guide instrument 14 frictionally secured within the bone tissue of the surgically-prepared proximal end 28 of the patient's femur 26, the surgeon grips the handle 30 and urges it distally toward the proximal guide instrument 14. Doing so advances the cement restrictor trial component 16 distally and thus further into the intramedullary canal 24 of the patient's femur 26. As shown in FIG. 6, during such advancement of the cement restrictor trial component 16, the surgeon may drastically bow the flexible elongated shaft 40 as the surgeon advances the shaft 40 “around the corner” and into the proximal end 28 of the patient's femur 26 as a result of not having line-of-sight access to the intramedullary canal 24 during a direct anterior approach hip arthroplasty.

The surgeon continues to advance the handle 30 (and hence the cement restrictor trial component 16) distally toward the proximal guide instrument 14 until the depth stop 44 engages the superior end 54 of the proximal guide instrument 14 thus preventing further advancement of the flexible elongated shaft 40 (and hence the cement restrictor trial component 16). Engagement of the depth stop 44 with the proximal guide instrument 14 provides an indication to the surgeon that the cement restrictor trial component 16 has reached the desired depth.

Once the cement restrictor trial component 16 has reached the desired depth, the surgeon then confirms that the cement restrictor trial component 16 is firmly seated in the intramedullary canal 24. If the surgeon is satisfied with the fit of the cement restrictor trial component 16 within the intramedullary canal 24, the surgeon pulls or otherwise urges the handle 30 and the flexible elongated shaft 40 in a direction away from the proximal guide instrument 14 thereby advancing the cement restrictor trial component 16 proximally within the intramedullary canal 24 in a direction toward the proximal guide instrument 14. When the cement restrictor trial component 16 reaches the proximal guide instrument 14, the surgeon removes the proximal guide instrument 14 (and hence the insertion instrument 12 and the cement restrictor trial component 16) from the surgically-prepared proximal end 24 of the patient's femur.

The surgeon then unthreads the cement restrictor trial component 16 from the threaded distal tip 34 of the insertion instrument 12 and replaces it with the cement restrictor implant component 18 by threading the implant component 18 onto the threaded distal tip 34.

The surgeon then utilizes the insertion instrument 12 and the proximal guide instrument 14 to install the cement restrictor implant component 18 at the desired depth within the intramedullary canal 24 of the patient's femur 26 in an identical manner to as discussed above in regard to the cement restrictor trial component 16 and illustrated in FIGS. 4-7. However, once the cement restrictor implant component 18 has been advanced to the desired depth within the intramedullary canal 24 of the patient's femur 26, the surgeon rotates the handle 30 of the insertion instrument 12 in a direction (e.g., counterclockwise) which unthreads the distal tip 34 from the cement restrictor implant component 18 thereby unsecuring the insertion instrument 12 from the implanted cement restrictor implant component 18. The surgeon then pulls or otherwise urges the handle 30 and the flexible elongated shaft 40 in a direction away from the proximal guide instrument 14 thereby advancing the freed threaded distal tip 34 proximally within the intramedullary canal 24 in a direction toward the proximal guide instrument 14. The surgeon then removes the insertion instrument 12 and the proximal guide instrument 14 from the patient's femur 26 and completes the hip replacement procedure.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. A method of installing a cement restrictor component during performance of a direct anterior approach orthopaedic surgical hip replacement procedure on a patient's femur, comprising:
sliding an elongated shaft of an insertion instrument through an elongated bore of a proximal guide instrument, the insertion instrument having a handle on its proximal end and the proximal guide instrument having an elongated post through which the bore extends and a neck extending medially from the elongated post,
securing the cement restrictor component onto a distal end of the insertion instrument so as to slidably capture the proximal guide instrument on the elongated shaft between the handle of the insertion instrument and the cement restrictor component,
advancing the cement restrictor component through a surgically-prepared proximal end of the patient's femur and into an intramedullary canal of the patient's femur,
sliding the proximal guide instrument along the shaft of the insertion instrument such that the post and the neck of the proximal guide instrument are frictionally secured in the surgically-prepared proximal end of the patient's femur, and
sliding the shaft of the insertion instrument relative to the secured proximal guide instrument such that the cement restrictor component is advanced distally into the intramedullary canal of the patient's femur to a desired depth.

2. The method of claim 1, further comprising:
unsecuring the cement restrictor component from the distal end of the insertion instrument once the cement restrictor component has been advanced to the desired depth, and
sliding the shaft of the insertion instrument relative to the secured proximal guide instrument such that the distal end of the insertion instrument is advanced proximally out of the intramedullary canal of the patient's femur.

3. The method of claim 1, wherein securing the cement restrictor component onto the distal end of the insertion instrument comprises threading the cement restrictor component onto a threaded end of the shaft of the insertion instrument.

4. The method of claim 1, wherein:
a superior end of the post of the proximal guide instrument defines a planar surface,
the shaft of the insertion instrument has a depth stop secured thereto at a location between the handle of the insertion instrument and the distal end of the insertion instrument,
a distal surface of the depth stop defines a planar surface, and
sliding the shaft of the insertion instrument relative to the secured proximal guide instrument such that the cement restrictor component is advanced distally into the intramedullary canal of the patient's femur to the desired depth comprises sliding the shaft of the insertion instrument relative to the secured proximal guide instrument until the planar surface of the depth stop engages the planar surface of the proximal guide instrument.

5. The method of claim 1, wherein:
the cement restrictor component comprises a cement restrictor trial component, and
sliding the shaft of the insertion instrument relative to the secured proximal guide instrument comprises sliding the shaft of the insertion instrument relative to the secured proximal guide instrument such that the cement restrictor trial component is advanced distally into the intramedullary canal of the patient's femur to the desired depth.

6. The method of claim 1, wherein:
the cement restrictor component comprises a cement restrictor implant component, and
sliding the shaft of the insertion instrument relative to the secured proximal guide instrument comprises sliding the shaft of the insertion instrument relative to the secured proximal guide instrument such that the cement restrictor implant component t is advanced distally into the intramedullary canal of the patient's femur to the desired depth.

7. The method of claim 1, wherein sliding the shaft of the insertion instrument relative to the secured proximal guide instrument comprises bowing the shaft of the insertion instrument during advancement of the cement restrictor component.

8. A method of installing a cement restrictor implant component during performance of a direct anterior approach orthopaedic surgical hip replacement procedure on a patient's femur, comprising:
sliding an elongated shaft of an insertion instrument through an elongated bore of a proximal guide instrument, the insertion instrument having a handle on its proximal end and the proximal guide instrument having an elongated post through which the bore extends and a neck extending medially from the elongated post,
securing the cement restrictor implant component onto a distal end of the insertion instrument so as to slidably capture the proximal guide instrument on the elongated shaft between the handle of the insertion instrument and the cement restrictor implant component,
advancing the cement restrictor implant component through a surgically-prepared proximal end of the patient's femur and into an intramedullary canal of the patient's femur,
sliding the proximal guide instrument along the shaft of the insertion instrument such that the post and the neck of the proximal guide instrument are frictionally secured in the surgically-prepared proximal end of the patient's femur,
sliding the shaft of the insertion instrument relative to the secured proximal guide instrument such that the cement restrictor implant component is advanced distally into the intramedullary canal of the patient's femur to a desired depth, and
unsecuring the cement restrictor implant component from the distal end of the insertion instrument once the cement restrictor implant component has been advanced to the desired depth so as to implant the cement restrictor implant component at the desired depth.

9. The method of claim 8, further comprising sliding the shaft of the insertion instrument relative to the secured proximal guide instrument such that the distal end of the insertion instrument is advanced proximally out of the intramedullary canal of the patient's femur.

10. The method of claim 8, wherein securing the cement restrictor implant component onto the distal end of the insertion instrument comprises threading the cement restrictor implant component onto a threaded end of the shaft of the insertion instrument.

11. The method of claim 8, wherein:
a superior end of the post of the proximal guide instrument defines a planar surface,
the shaft of the insertion instrument has a depth stop secured thereto at a location between the handle of the insertion instrument and the distal end of the insertion instrument,
a distal surface of the depth stop defines a planar surface, and
sliding the shaft of the insertion instrument relative to the secured proximal guide instrument such that the cement restrictor implant component is advanced distally into the intramedullary canal of the patient's femur to the desired depth comprises sliding the shaft of the insertion instrument relative to the secured proximal guide instrument until the planar surface of the depth stop engages the planar surface of the proximal guide instrument.

12. The method of claim 8, wherein sliding the shaft of the insertion instrument relative to the secured proximal guide instrument comprises bowing the shaft of the insertion instrument during advancement of the cement restrictor implant component.

13. An orthopaedic surgical system for use in a direct anterior approach orthopaedic surgical hip replacement procedure on a patient's femur, comprising:
a proximal guide instrument having (i) an elongated post having a bore formed therein, the bore extending from a superior end of the elongated post to an inferior end of the elongated post and having a common diameter throughout its entire length, and (ii) a neck extending medially from the elongated post,
an insertion instrument having (i) a handle at its proximal end, (ii) a threaded distal end, and (iii) a flexible elongated shaft extending distally away from the handle to the threaded distal end, the flexible elongated shaft having a diameter that is sized to be slidingly received in the bore of the proximal guide instrument, and
a cement restrictor component configured to be threaded onto the threaded distal end of the insertion instrument.

14. The orthopaedic surgical system of claim 13, wherein the flexible elongated shaft is constructed of a superelastic metal.

15. The orthopaedic surgical system of claim 13, wherein the flexible elongated shaft is constructed of a nickel titanium alloy.

16. The orthopaedic surgical system of claim 13, wherein the cement restrictor component comprises a cement restrictor trial component.

17. The orthopaedic surgical system of claim 13, wherein the cement restrictor component comprises a cement restrictor implant component.

18. The orthopaedic surgical system of claim 13, wherein,
the superior end of the post of the proximal guide instrument defines a planar surface,
the flexible elongated shaft of the insertion instrument has a depth stop secured thereto at a location between the handle of the insertion instrument and the threaded distal end of the insertion instrument, and
a distal-most surface of the depth stop defines a planar annular surface having a diameter that is larger than the common diameter of the bore of the proximal guide instrument.
